(19) United States
(12) Patent Application Publication
(10) Pub. No.: US 2014/0172104 A1
(43) Pub. Date: Jun. 19, 2014

(54) INSTRUMENT FOR INSERTION AND DEPLOYMENT OF AN IMPLANT

(71) Applicant: Alphatec Spine, Inc., Carlsbad, CA (US)

(72) Inventors: Amardeep DUGAL, San Marcos, CA (US); Yang CHENG, Foothill Ranch, CA (US); Nirali PATEL, Carlsbad, CA (US); Budi BUDIKENTJANA, San Diego, CA (US)

(21) Appl. No.: 13/718,092

(22) Filed: Dec. 18, 2012

(51) Int. Cl. A61F 2/46; A61F 2/44

(52) U.S. Cl. CPC A61F 2/4611; A61F 2/442
USPC 623/17.16; 606/86 A

(57) ABSTRACT

An exemplary surgical instrument for inserting a spinal implant includes an inserter portion, a coupling member, a first actuator, and a second actuator. The inserter portion includes at a proximal end a receiving block, at a distal end a sleeve, and a channel extending therethrough. The coupling member includes at a proximal end movably coupled with the receiving block, a middle portion that slides within the channel, a distal end that couples the spinal implant, and a first axial bore extending therethrough. The first actuator includes a proximal end with a first projection for engaging the receiving block, a first shaft extending through the first axial bore, and a distal end with a first engagement feature for engaging a first deployment feature of the implant. The second actuator includes a proximal lend with a second projection for engaging the receiving block, a second shaft extending through a second axial bore of the first shaft, and a distal end with a second engagement feature for engaging a second deployment feature of the implant.

### CROSS-REFERENCE TO RELATED APPLICATIONS

The present application is a continuation-in-part of U.S. patent application Ser. No. 12/944,507 to Patel et al. filed Nov. 11, 2010, which claims priority to U.S. Provisional Application 60/260,323 to the same inventors filed Nov. 11, 2009, both incorporated by reference in their entirety.

### FIELD

The present disclosure generally relates to the field of spinal orthopedics, and more particularly to instruments for insertion and deployment of features of an implant.

### BACKGROUND

The spine is a flexible column formed of a plurality of bones called vertebra. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibrocartilaginous bodies.

The intervertebral fibro-cartilages are also known as intervertebral disks and are made of a fibrous ring filled with pulpy material. The disks function as spinal shock absorbers and also cooperate with synovial joints to facilitate movement and maintain flexibility of the spine. When one or more disks degenerate through accident or disease, nerves passing near the affected area may be compressed and are consequently irritated. The result may be chronic and/or debilitating back pain.

Various methods and apparatus have been designed to relieve such back pain, including spinal fusion using an interbody spacer or suitable graft using techniques such as Anterior Lumbar Interbody Fusion (ALIF), Posterior Lumbar Interbody Fusion (PLIF), or Transforaminal Lumbar Interbody Fusion (TLIF) surgical techniques. The implants used in these techniques, also commonly referred to as vertebral body replacements (VBR) devices, are placed in the interdiscal space between adjacent vertebrae of the spine. Many times an exterior plate is used in conjunction with the VBR to hold the adjacent vertebrae while the fusion occurs. Some implants may include features for attachment to the vertebrae. These features may be deployed using one or more instruments.

### SUMMARY

An exemplary surgical instrument for inserting a spinal implant includes an inserter portion, a coupling member, a first actuator, and a second actuator. The inserter portion includes at a proximal end a receiving block, at a distal end a sleeve, and a channel extending therethrough. The coupling member includes at a proximal end movably coupled with the receiving block, a middle portion that slides within the channel, a distal end that couples the spinal implant, and a first axial bore extending therethrough. The first actuator includes a proximal end with a first projection for engaging the receiving block, a first shaft extending through the first axial bore, and a distal end with a first engagement feature for engaging a first deployment feature of the implant. The second actuator includes a proximal lend with a second projection for engaging the receiving block, a second shaft extending through a second axial bore of the first shaft, and a distal end with a second engagement feature for engaging a second deployment feature of the implant.

In other features, the receiving block includes features for limiting rotational movement of at least one of the first actuator and the second actuator. The receiving block includes an initial slot on the circumferential surface at a first radius from a longitudinal axis of the instrument. The initial slot transitions to a ramped portion of increasing radius. The ramped portion terminates at a second radius that is great than the first radius. The ramped portion transitions to a deployed slot at a radius less than the second radius. The first projection is configured to engage at least one of the initial slot, the ramped portion, and the deployed slot during actuation of the first deployment feature of the implant.

In still other features, the proximal end of the first actuator includes a receiving end with features for limiting rotational movement of at least one of the first actuator and the second actuator. The receiving end includes a first notch on the circumferential surface at a first angle relative to a first handle assembly. The receiving end includes a second notch disposed at a second angle relative to the first handle assembly. The receiving end includes a ramped portion between the first notch and the second notch.

In yet other features, the instrument includes a release mechanism that forces the first actuator and the second actuator apart. The release mechanism includes a lever arm and a cam portion coupled with the second actuator such that rotation of the lever arm applies force through the cam portion on the first actuator.

In still yet other features, the coupling member includes a pair of arms at the distal end for clamping the spinal implant. The first actuator rotates in a first direction to deploy the first deployment feature and the second actuator rotates in a second direction to deploy the second deployment feature. The distal end of the first shaft and the distal end of the second shaft includes at least one of a splined projection and a hexagonal projection.

An exemplary system for a spinal procedure includes an implant and an instrument. The implant includes a first deployment feature and a second deployment feature configured to engage with vertebrae. The instrument includes an inserter portion, a coupling member, a first actuator, and a second actuator. The inserter portion includes at a proximal end a receiving block, at a distal end a sleeve, and a channel extending therethrough. The coupling member includes at a proximal end movably coupled with the receiving block, a middle portion that slides within the channel, a distal end that couples the spinal implant, and a first axial bore extending therethrough. The first actuator includes a proximal end with a first projection for engaging the receiving block, a first shaft extending through the first axial bore, and a distal end with a first engagement feature for engaging a first deployment feature of the implant. The second actuator includes a proximal lend with a second projection for engaging the receiving block, a second shaft extending through a second axial bore of the first shaft, and a distal end with a second engagement feature for engaging a second deployment feature of the implant.

In other features, the receiving block includes features for limiting rotational movement of at least one of the first actuator and the second actuator to limit actuation of at least one of the first deployment feature and the second deployment feature. In yet other features, the proximal end of the first actuator includes a receiving end with features for limiting rotational movement of at least one of the first actuator and the second actuator. In still yet other features, the instrument includes a release mechanism that forces the first actuator and the second actuator apart.

### BRIEF DESCRIPTION OF THE DRAWINGS

FIG. 1 is a perspective view of an exemplary surgical instrument and implant according to the principles of the present disclosure and an implant.

FIG. 2 is a partially exploded perspective view of the surgical instrument according to the principles of the present disclosure.

FIG. 3 is an exploded perspective view of a housing and a first shaft of the surgical instrument according to the principles of the present disclosure.

FIG. 4 is an exploded perspective view of a handle assembly and a second shaft of the surgical instrument according to the principles of the present disclosure.

FIG. 5 is a top elevational view of a sleeve of the surgical instrument according to the principles of the present disclosure.

FIGS. 6 and 7 are top and side elevational views of a clamping member of the surgical instrument according to the principles of the present disclosure.

FIGS. 8-10 are partial perspective views of a distal end of the surgical instrument engaging and deploying features of the implant according to the principles of the present disclosure.

FIG. 11 is a perspective view of a second exemplary instrument according to the principles of the present disclosure.

FIG. 12 is a partially exploded perspective view of the second instrument according to the principles of the present disclosure.

FIGS. 13-16 are perspective, top elevational, and side views of a receiving block of the second instrument according to the principles of the present disclosure.

FIG. 17 is a perspective view of a proximal end of an outer shaft and handle assembly of the second instrument according to the principles of the present disclosure.

FIG. 18 is a perspective view of a proximal end of an inner shaft and handle assembly of the second instrument according to the principles of the present disclosure.

FIGS. 19 and 20 are perspective views of the second instrument illustrating actuation for deploying the deployment features of the implant.

### DETAILED DESCRIPTION

Implants or vertebral body replacements may be placed in the interdiscal space between adjacent vertebrae of the spine. The implants may include one or more features that are deployed to affix the implants to the adjacent vertebrae. For example, a stand-alone interbody fixation system, as disclosed in commonly assigned U.S. continuation-in-part application Ser. No. 13/633,301 to Vishnubhotla et al. filed Oct. 2, 2012, which is a continuation-in-part of U.S. Pat. No. 8,328,870, to Patel et al., both of which are incorporated by reference in their entirety, provides a solid fixation in all aspects by using counter-rotating blades that provide fixation to the vertebrae. Such implants may be inserted, positioned, and deployed by various embodiments of the instrument of the present disclosure. As can be understood by one skilled in the art, these embodiments are shown for illustrative purposes and are not intended to limit the scope of the invention.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The words proximal and distal are applied herein to denote specific ends of components of the instrument described herein. A proximal end refers to the end of an instrument nearer to an operator of the instrument when the instrument is being used. A distal end refers to the end of the instrument further from the operator and extending towards the surgical area of a patient and/or the implant.

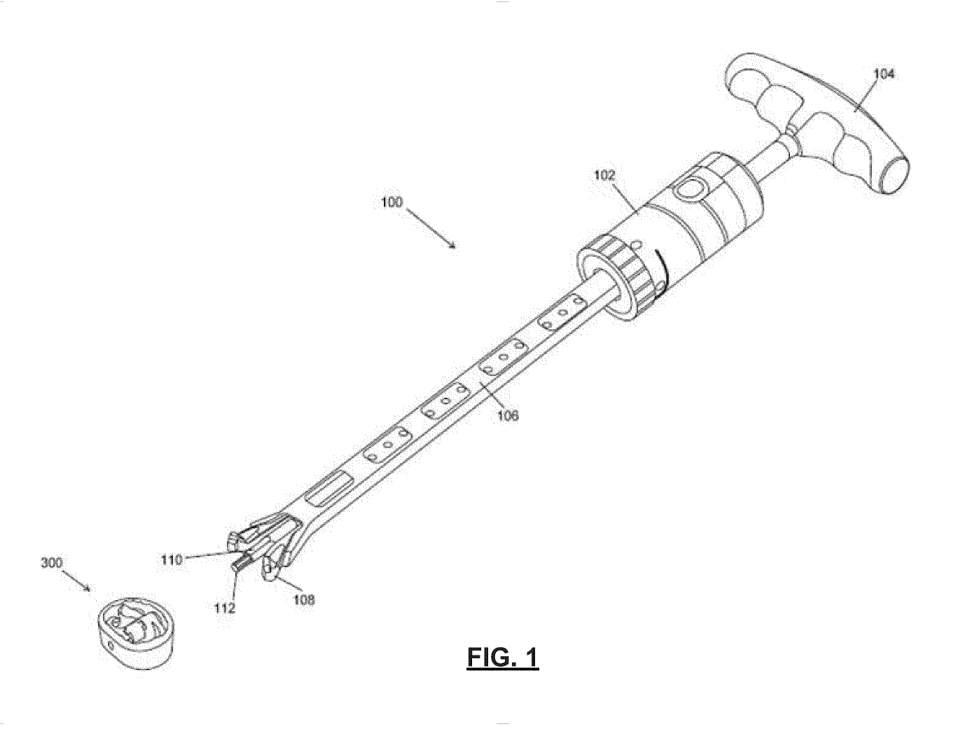

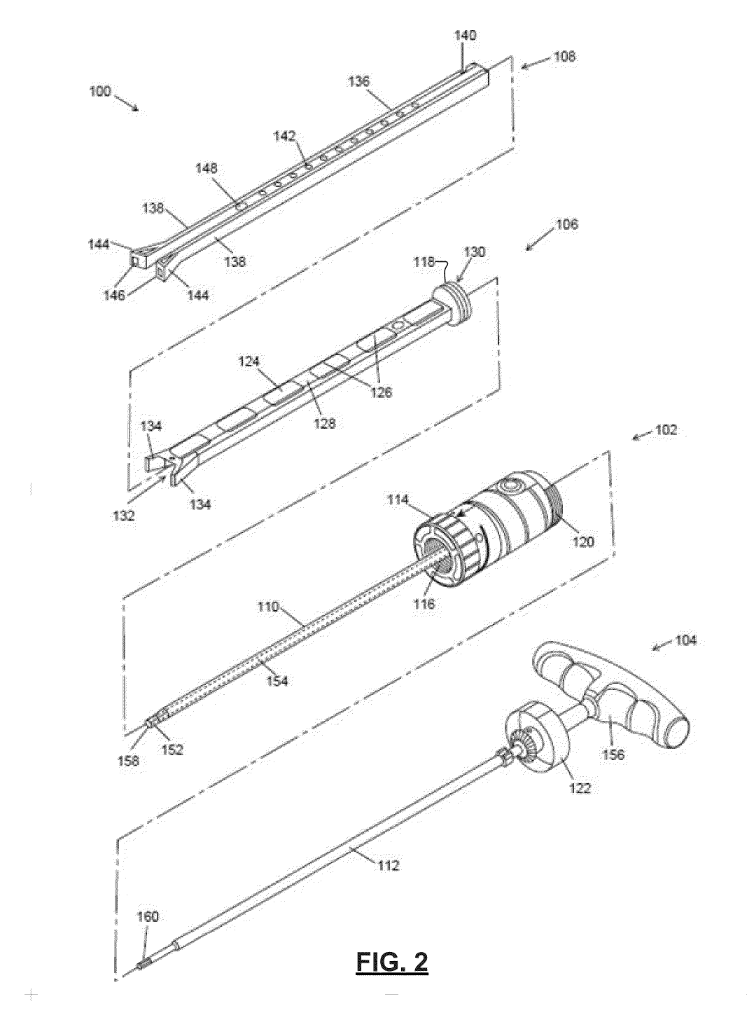

Referring to FIGS. 1 and 2, a surgical instrument 100 according to the principles of the present disclosure is shown. The instrument 100 includes features to enable insertion of an implant 300 into the spinal area of a patient and to enable actuation of additional features of the implant 300 after insertion. The instrument includes a housing 102, a handle assembly 104, a sleeve 106, a clamping member 108, an outer shaft 110, and an inner shaft 112. The clamping member 108 includes features that enable coupling of the instrument 100 with the implant 300. The sleeve 106 includes features that actuate the coupling member 108 to couple the implant 300. When the implant 300 is coupled, the instrument 100 may be used to position the implant 300 in the spinal area of the patient. The outer shaft 110 and the inner shaft 112 include features that actuate the additional features of the implant 300. For example, once the implant 300 has been positioned in the spinal area, the outer shaft 110 and the inner shaft 112 may be rotated to deploy features of the implant 300 that engage with vertebrae in the spinal area.

Referring now to FIG. 2, a partial exploded view shows additional features of the instrument 100. The housing 102 includes features for attachment of the sleeve 106, the clamping member 108, the outer shaft 110, and the handle assembly 104. The housing 102 includes a wheel 114 that freely rotates on the distal end of the housing 102 and includes features that engage with the sleeve 106. For example, the wheel 114 may include a thread 116 on an inner surface of the wheel 114 that engages with a corresponding threaded portion 118 on the proximal end of the sleeve 106. The housing 102 may include a threaded portion 120 at the proximal end for attachment of the handle assembly 104. For example, the threaded portion 120 may engage with threads of a cap 122 on the handle assembly 104.

The sleeve 106 includes a channel 124 that extends the length of the sleeve 106 and is configured to receive the clamping member 108. The channel 124 may be formed by boring a first series of windows 126 on a first side of the sleeve. The windows 126 may be cut into the sleeve 106 until a wall of material remains on a second side of the sleeve opposite the first side. After the first series of windows 126 has been cut, a second series of windows may be cut through the second side of the sleeve until cross members 128 of material remain on the first side of the sleeve. The second series of windows may be spaced to remove remaining material between the first series of windows 126. The resulting channel 124 is configured to permit sliding engagement with the clamping member 108.

The sleeve 106 forms an opening 130 at the proximal end to permit passage of the clamping member 108 through the channel 124 into the housing 102. The sleeve 106 forms a mouth 132 at the distal end that allows for sliding engagement with the clamping member 108. The mouth 132 may include flared portions 134 that flare away from a center line of the instrument 100. The flared portions 134 are configured to engage with the clamping member 108 and to couple the implant 300 to the instrument 100 as described below.

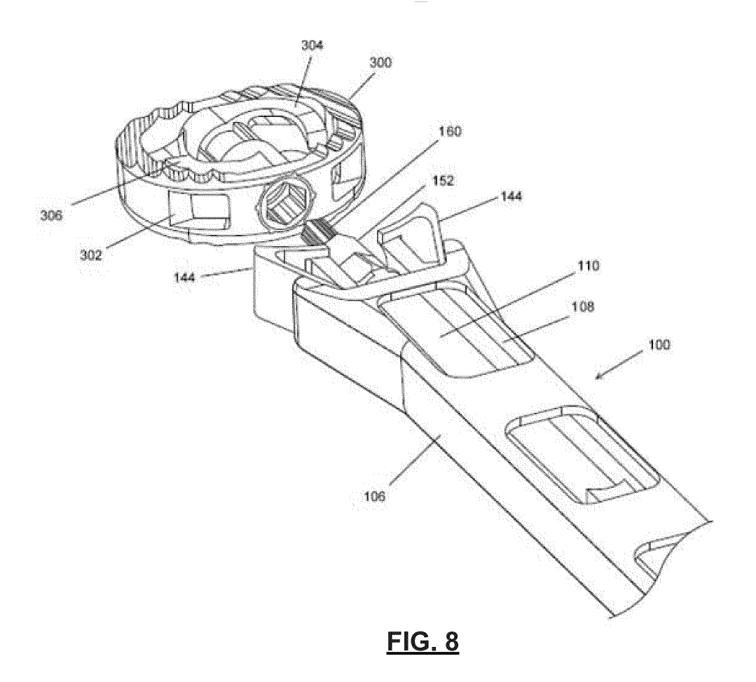

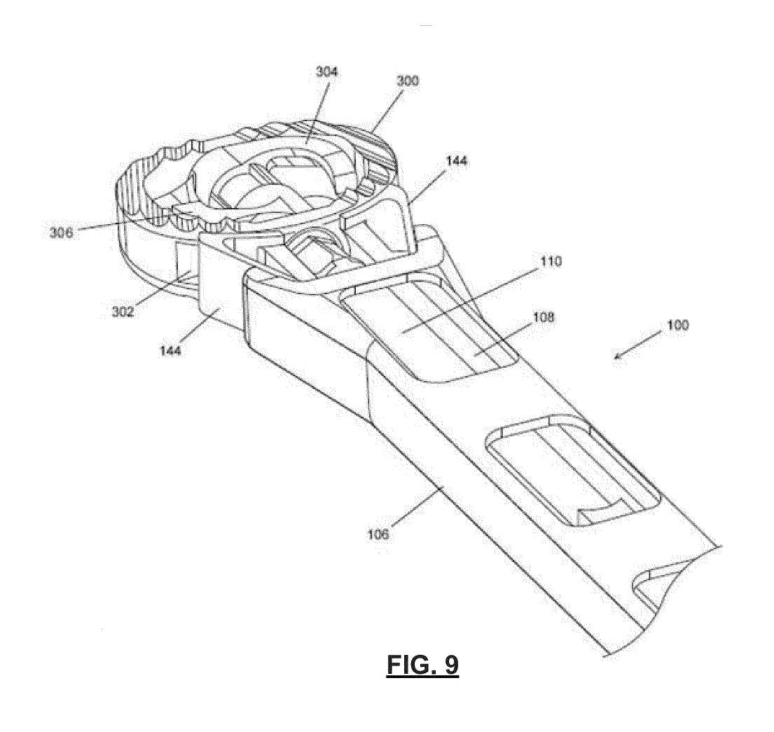

Continuing with FIG. 2, the clamping member 108 includes an elongated portion 136 and a pair of arms 138. The elongated portion 136 extends through the channel 124 to engage with the housing 102. At the proximal end of the elongated portion 136, a locking feature 140 is configured to fixedly attach the clamping member 108 to the housing 102. For example the locking feature 140 may include a notch cut into the clamping member 108 that engages with a lock pin (not shown) inside the housing 102. The elongated portion 136 may further include one or more holes 142 to facilitate cleaning of the instrument 100 and to reduce the weight. The pair of arms 138 extends from the distal end of the elongated portion 136 and terminates in a pair of tips 144. The tips 144 may flare away from the center line of the instrument 100 similar to the flared portions 134 of the sleeve 106. The tips 144 may include projections 146 that partially extend towards the center line of the instrument 100. In other features, the projections 146 may partially extend towards the distal end of the instrument 100. The clamping member 108 further includes a first axial bore 148 that extends the length of the elongated portion 136 to enable the outer shaft 110 to pass through the clamping member 108 as depicted in FIGS. 8 and 9.

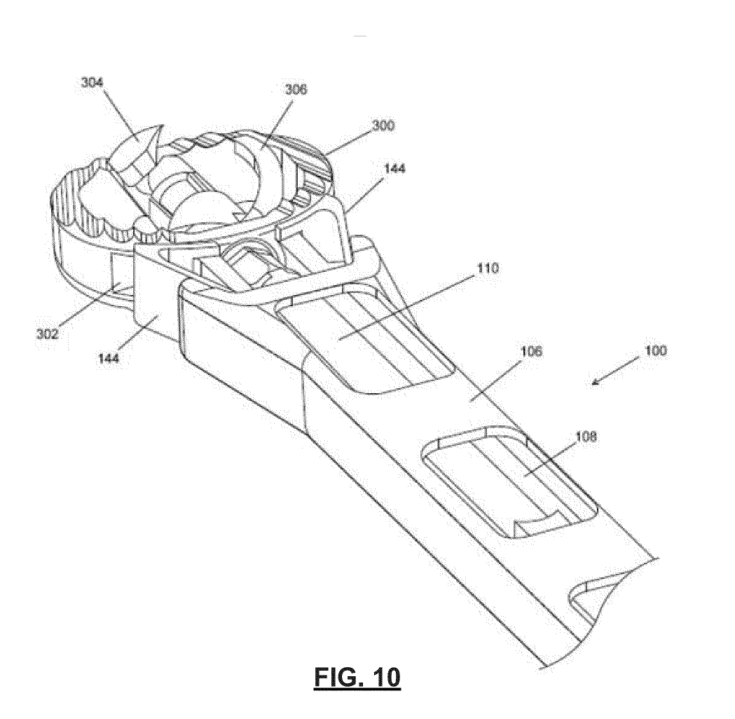

Once the sleeve 106 and clamping member 108 are coupled within the housing 102 and the wheel 114 on the housing is rotated, the threaded portion 118 of the sleeve 106 advances along the thread 116 of the wheel 114 causing the sleeve 106 to slide relative to the clamping member 108. For example, rotating the wheel 114 in a first direction advances the sleeve 106 towards the distal end of the instrument 100 while rotating the wheel in a second direction retracts the sleeve 106 towards the proximal end of the instrument 100. As the sleeve 106 advances towards the distal end of the instrument 100, the flared portions 134 engage with the tips 144. The flared portions 134 compress the tips 144 towards the center line of the instrument 100 causing the arms 138 to flex inwardly towards the center line. As shown in FIG. 10, the tips 144 are configured to engage with corresponding holes 302 on the implant 300. As the tips 144 are compressed, the projections 146 provide a clamping force on the implant 300 to couple the implant 300 to the instrument 100.

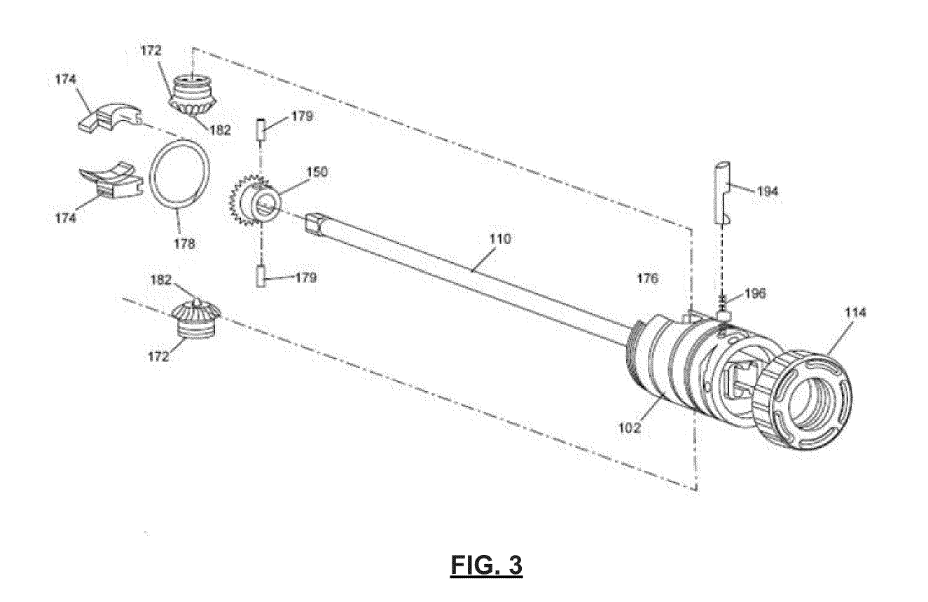

Continuing with FIG. 2, the outer shaft 110 extends from the housing 102 through the first axial bore 148 in the clamping member 108. The outer shaft 110 may freely rotate in the first axial bore 148 relative to the clamping member 108. Referring to FIGS. 2 and 3, the proximal end of the outer shaft 110 may couple with a follower gear 150 inside the housing 102 and the distal end extends through the axial bore 148 and past the mouth 132 of the sleeve 106. The distal end of the outer shaft 110 includes a first engagement feature 152 configured to actuate a first deployment feature, such as a first blade 304 of the implant 300 as depicted in FIGS. 10-12. For example, the first engagement feature 152 may include a hexagonal projection. The outer shaft 110 further includes a second axial bore 154 extending through the length of the outer shaft 110 from the proximal end to the distal end.

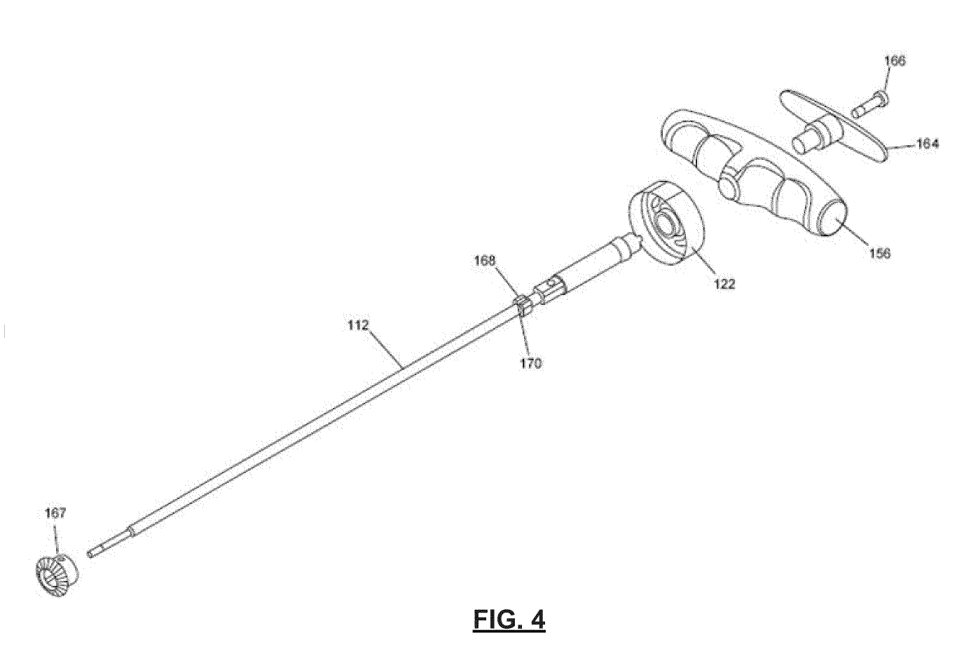

Referring now to FIGS. 2 and 4, the inner shaft 112 extends from the handle assembly 104 through the second axial bore 154 in the outer shaft. The inner shaft 112 freely rotates in the second axial bore 154 relative to the outer shaft 110. The proximal end of the inner shaft 112 may couple with a handle 156 and a distal end may extend past an opening 158 in the first axial bore 148. The distal end of the inner shaft 112 includes a second engagement feature 160 configured to actuate a second deployment feature, such as a second blade 306 of the implant as depicted in FIGS. 10-12. For example, the second engagement feature 160 may include a splined projection.

Referring now to FIGS. 3 and 4, exploded views of the handle assembly 104, inner shaft 112, housing 102, and outer shaft 110 are shown in greater detail. The handle assembly 104 includes the cap 122, the handle 156, and a striking member 164. The inner shaft 112 may pass though the cap 122 and the handle 156 to connect with the striking member 164 using a screw 166 or other fixation member. The cap 122 couples the handle assembly 104 to the housing 102 and allows rotation of the handle 156 and inner shaft 112. When the operator applies torque to rotate the handle 156, the inner shaft 112 rotates together and in the same direction as the handle 156. In addition, the operator may apply a striking force to the striking member 164 to drive the implant 300 further into the spinal area. The striking member 164 transfers the striking force to the inner shaft 112.

A drive gear 167 and an alignment block 168 are disposed along the proximal end of the inner shaft 112. The drive gear 167 may be fixedly attached to the inner shaft 112 to rotate with the inner shaft 112. The alignment block 168 may be fixedly attached to or formed on the inner shaft 112 and positioned to align the inner shaft 112 and the outer shaft 110 during assembly of the instrument 100. The alignment block 168 may include notches 170 to align the inner shaft 112 relative to the outer shaft 110 as described below. In addition, when the operator applies a striking force to the striking member 164, the alignment block 168 may transfer the striking force to the follower gear 150 and the housing 102 rather than to the distal end of the inner shaft 112. The housing 102 then transfers the striking force to the sleeve 106 to position the implant 300 deeper into the spinal area. This may prevent damage to the engagement features of the shafts.

As depicted in FIG. 3, the housing 102 includes a gear set that transfers torque from the drive gear 167 to the outer shaft 110. The gear set includes one or more transfer gears 172 configured to be driven by the drive gear 167 on the inner shaft 112. The transfer gears 172 may be, for example, beveled gears. The transfer gears 172 may be rotatably fixed in removable sleeves 174 disposed in U-shaped channels 176 of the housing 102. A clip 178 may lock the sleeves 174 into the U-shaped channels 176.

When the operator applies torque to the handle 156, the drive gear 167 rotates in the same direction as the handle 156. The inner shaft 112, also attached to the handle 156, rotates in the same direction. The drive gear 167 causes the transfer gears 172 to rotate and transfer torque to the follower gear 150 attached to the proximal end of the outer shaft 110. The transfer gears 172 cause the follower gear 150 to rotate in the opposite direction as the drive gear 167. Thus, the outer shaft 110, which is attached to the follower gear 150, rotates in the opposite direction as the inner shaft 112. Thus, when the operator rotates the handle 156 in one direction, the inner shaft 112 and the outer shaft 110 counter-rotate.

One or more anti-rotation pins 179 may be used attach the follower gear 150 and to prevent the inner shaft 112 and the outer shaft 110 from rotating beyond a predetermined angle. For example, the anti-rotation pins 179 may radially extend away from the center line of the instrument 100 in a cavity 180 of the housing 102. The cavity 180 may prevent rotation of the pins 179 beyond the predetermined angle. For example, the anti-rotation pins 179 may prevent over-rotation of the first and second deployment features 304, 306 of the implant 300.

The transfer gears 172 may also include one or more alignment nibs 182 that extend from the center of the transfer gears 172 towards the inner shaft 112. The alignment nibs 182 may be used in conjunction with the notches 170 of the alignment block 168 to align the inner shaft 112 with the outer shaft 110 during assembly. For example, the inner shaft 112 may not extend through the opening 158 unless the alignment nibs 182 are lined up with the notches 170. When the alignment nibs 182 pass through the notches 170, then the first and second engagement features 152 and 160 are properly aligned for engagement with corresponding features on the implant 300.

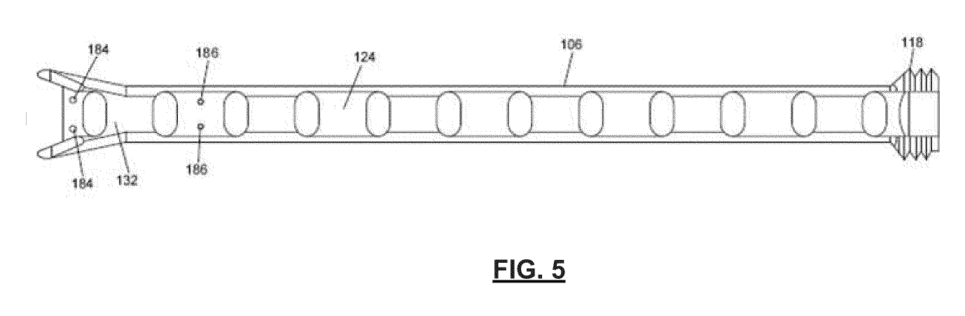

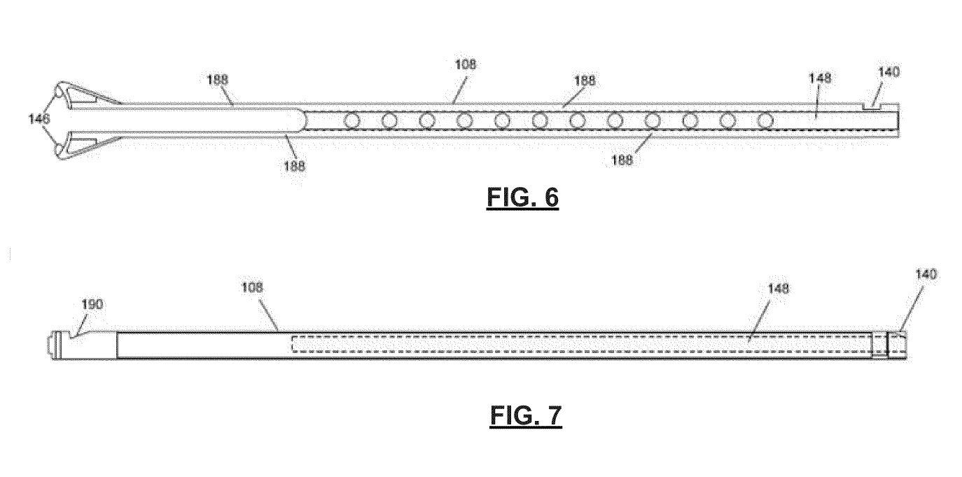

Referring now to FIGS. 5-7 the sleeve 106 and clamping member 108 are shown in greater detail. The sleeve 106 may include a first set of guide pins 184 on an inner surface of the mouth 132. The sleeve 106 may include a second set of guide pins 186 on an inner surface of the channel 124. The guide pins 184, 186 slidably engage with a track 188 on the clamping member 108. The guide pins 184, 186 prevent over-deflection of the arms 138 when the sleeve 106 advances towards the distal end of the clamping member 108. Additional guide pins may be provided at other locations inside the channel 124 to engage with the track 188 and maintain structural integrity of the clamping member 108.

The clamping member 108 may include a positioning marker 190 on the tips 144 to aid in locating the position of the implant 300 relative to the spinal area under fluoroscopy. The positioning marker 190 may be a cutout or notch in a sidewall of the tips 144. In additional features, the clamping member 108 may attach to the distal end of the housing 102 using the locking feature 140. For example, the clamping member 108 may be inserted into a receptacle in the distal end of the housing 102 as seen in FIG. 3. The locking feature 140 may then engage with a lock pin 194 disposed in the housing 102. The lock pin 194 may include a spring 196 to facilitate locking and release of the lock pin 194 from the locking feature 140.

Referring to FIGS. 8-10, the instrument 100 may be used to couple the implant 300 to the distal end of the instrument 100 and actuate features of the implant 300. In FIG. 8, the distal ends of the first shaft 110 and the second shaft 112 project from the axial bore 148 in the clamping member 108 so that the first and second engagement features 152 and 160 may engage with corresponding features of the implant 300. The sleeve 106 is retracted so that the flared portions 134 do not compress the tips 144 of the clamping member 108. Thus, the projections 146 on the tips 144 may engage with the holes 302 in the implant 300. In FIG. 9, the projections 146 are engaged with the implant 300 and the sleeve 106 may be advanced towards the distal end of the instrument 100 using the wheel 114. The coupling member 108 holds the implant 300 to the outer shaft 110 and the inner shaft 112, enabling the first and second engagement features 152 and 160 to engage with corresponding receptacles in the implant 300. In FIG. 10, the operator applies torque to rotate the handle 156 which causes the inner shaft 112 to rotate and actuate the first deployment feature 304 of the implant. The gear set coupling the inner shaft 112 and the outer shaft 110 transfers torque from the inner shaft 112 to the outer shaft 110. The outer shaft 110 thus rotates in the opposite direction to actuate the second deployment feature 306 of the implant 300.

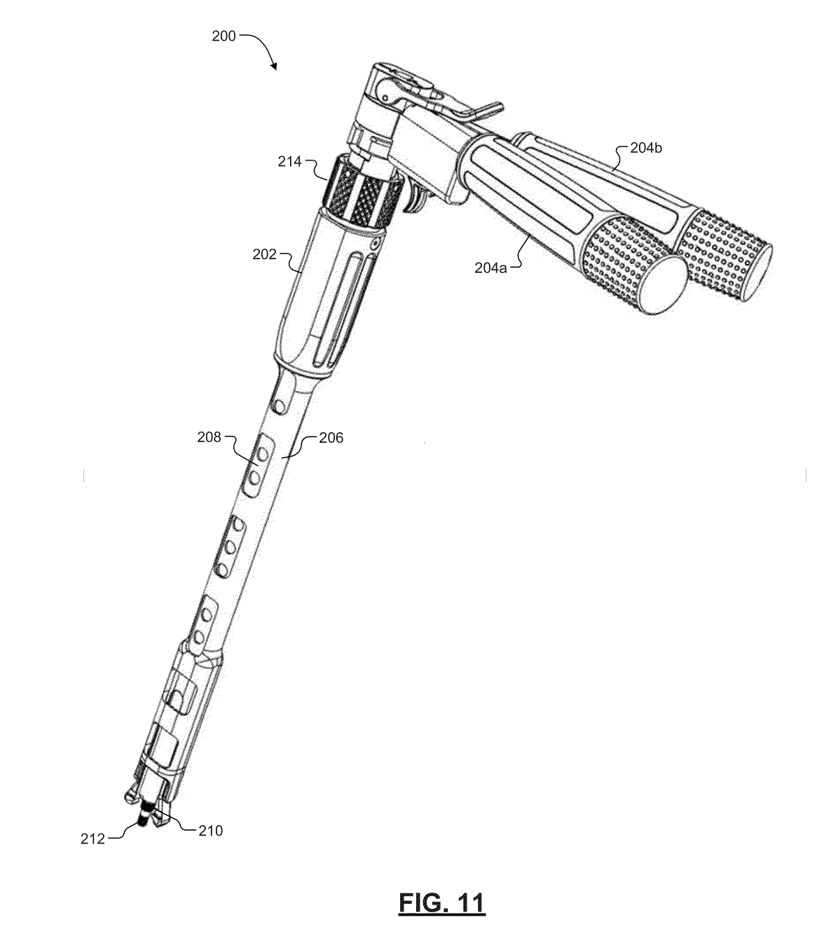

Referring now to FIG. 11, another exemplary surgical instrument 200 includes features to enable insertion of the implant 300 into the spinal area of a patient and to enable actuation of the deployment features of the implant 300 after insertion. The instrument 200 shares similar features as the first exemplary instrument 100 such as housing 102, handle 104, sleeve 106, and clamping member 108 as well as the counter-rotating outer shaft 110 and inner shaft 112. Therefore, similar numerals are used with reference to similar features. For example, the instrument 200 includes a housing 202, handle assemblies 204a and 204b (collectively handle assemblies 204), a sleeve 206, a clamping member 208, an outer shaft 210, and an inner shaft 212.

The instrument 200 includes features that enable coupling of with the implant 300 in a similar fashion as instrument 100. For example, the instrument 200 includes a positioning wheel 214 similar to the wheel 114. However, the sleeve 206 may not move in the present exemplary instrument 200 but may be rigidly secured to the housing 202. For example, the positioning wheel 214 may act on a proximal threaded end (not shown) of the clamping member 208 extending through the housing 202 in order to position the clamping member 208 within the attached sleeve 206. The sleeve 206 includes features that force portions of the clamping member 208 together to clamp the implant 300 similar to the sleeve 106. When the implant 300 is coupled to the clamping member 208, the instrument 200 may be used to position the implant 300 in the spinal area of the patient similar to instrument 100.

The outer shaft 210 and the inner shaft 212 include features that actuate the blades 304 and 306 of the implant 300. For example, once the implant 300 has been positioned in the spinal area, the outer shaft 210 and the inner shaft 212 may be rotated to deploy features of the implant 300 that engage with vertebrae in the spinal area. The distal ends of the shafts 210 and 212 may include various driving features including hexalobe, star-shaped, Allen-wrench, spline drives or any other suitable pattern that mates with features of the implant 300. In some examples, the distal ends of the shafts 210 and 212 may include one or more detachable tips such as inner shaft tip 260 and outer shaft tip 252 respectively as shown in FIG. 12. The detachable tips may deform under a predetermined amount of torque to prevent over deployment or rotation of the blades 304 or 306. The detachable tips may include various sizes for various sizes of implants 300. The detachable tips may be disposable or reusable.

Continuing now to FIG. 12, a partial exploded view shows additional features of the instrument 200. The housing 202 may include features for attachment of the sleeve 206 and/or the clamping member 208. In some examples, the sleeve 206 may be integral with the housing 202. The outer shaft 210 and the inner shaft 212 may pass through the housing 202 similar to the shafts 110 and 112 and housing 102. For example, the outer shaft 210 may include an outer diameter smaller than the diameter of a cannula or channel extending through the housing 202. The outer shaft 210 may include an axial bore similar to the axial bore 154 of instrument 100. The inner shaft 212 may be inserted through the axial bore of the outer shaft 210 and rotate freely therein.

The housing 202 may include the positioning wheel 214 on a proximal end which freely rotates and includes features that engage the proximal end of the clamping member 208 to position the clamping member 208 within the sleeve 206. For example, an inner surface of the wheel 214 may include a thread (not shown) that engages with a corresponding threaded portion (not shown) on the proximal end of the clamping member 208 similar to the thread 118 on the proximal end of the sleeve 106 of the instrument 100. Rotation of the wheel 214 may position the clamping member 208 relative to the sleeve 206. For example, as the wheel 214 rotates in one direction, the inner thread of the wheel 214 engages more of the threaded portion on the clamping member 208 and pulls the clamping member 208 proximally into the sleeve 206 to clamp the implant 300. Various features may be used to pull the clamping member 208 into the sleeve 206 to retain the implant 300 as described above with reference to the instrument 100. Rotation of the wheel 214 in the opposite direction positions the clamping member 208 distally to release the implant 300.

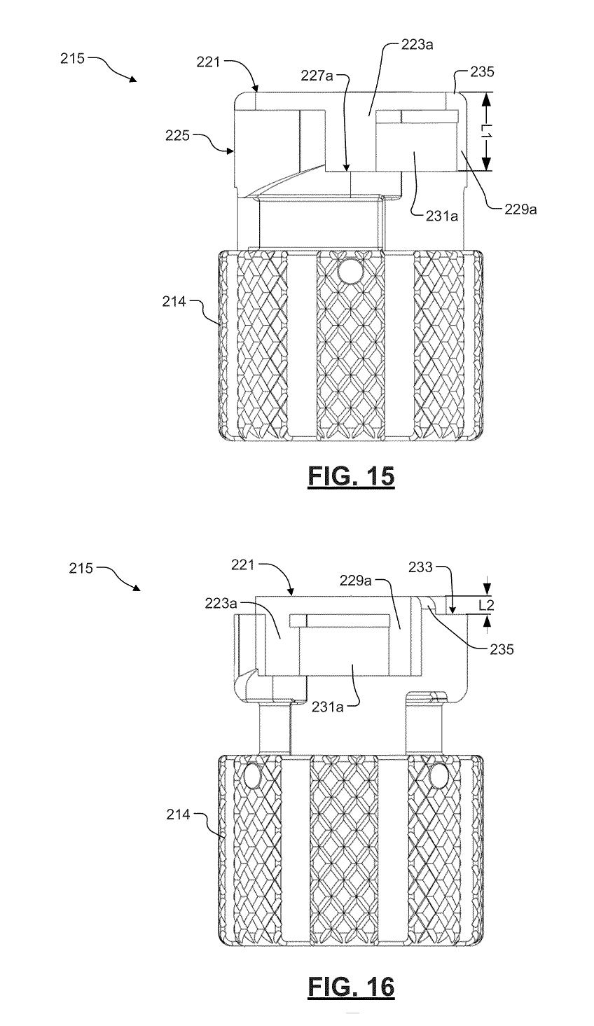

The proximal end of the housing 202 may include or couple with a receiving block 215 that receives the outer shaft 210. The receiving block 215 may include a generally cylindrical, cannulated shape from its proximal end to its distal end. Referring now also to FIGS. 13-16, the outer shaft 210 may be inserted through an aperture, channel, or cannula 217 in the block 215 extending from a proximal end to a distal end. The distal end may couple with the housing 202 through various means including welding, threaded connection, pins, etc. either removably or permanently. The receiving block 215 may include markings 219a and 219b (collectively markings 219) on a proximal surface 221 that assist with aligning the handle assemblies 204 during a deployment procedure.

The receiving block 215 may include various features that act on the handle assemblies 204 to align and/or limit rotation of the outer shaft 210 and the inner shaft 212. These features may control a deployment angle between the concentric shafts as described herein. Thus, actuation of the deployment features 304 and 306 of the implant 300 may be limited to prevent over-rotation. Further, full actuation of the deployment features 304 and 306 may correspond to tactile feedback provided by the features. For example, the receiving block 215 may include a plurality of features such as markings, slots, ramps, and tabs that aid with alignment of the handle assemblies 204 and engage features of the handle assemblies 204 to guide and limit rotation of the shafts 210 and 212 as described below. The plurality of features above may be repeated along the circumference of the receiving block 215. For example, the features may be repeated 180 degrees apart from one another.

For purposes of illustration, most features will be primarily discussed with reference to a first set of the features denoted with numerals ending in suffix “a.” A second set of the features, each of which may be repeated 180 degrees from the first set of features about the circumference of the receiving bock 215, will be denoted with numerals ending in suffix “b.” Features of the receiving block 215 may include the markings 219, the first surface 221, initial slots 223, an outer surface 225, first ledges 227, deployed slots 229, ramped portions 231, second ledges 233, and stops 235.

A first marking 219a may indicate a desired initial position of the first handle assembly 204a prior to deployment of the implant 300. A second marking 219b may indicate an initial position of the second handle 204b prior to deployment of the implant 300. The first marking 219a may be diametrically opposed 180 degrees from the second marking 219b. Either of the handle assemblies 204a and 204b may initially be aligned with either of the markings 219a and 219b. For example, the first handle assembly 204a may initially align with the first marking 219a or the second marking 219b. Likewise, the second handle assembly 204b may initially align with the first marking 219a or the second marking 219b. For purposes of clarity, the instrument 200 will be described with the first handle assembly 204a initially aligned with the first marking 219a and the second handle assembly 204b initially aligned with the second marking 219b.

Each of the markings 219 may be radially aligned with first and second initial slots 223a and 223b (collectively initial slots 223) on a proximal portion of the receiving block 215 that engage features of at least one of the handle assemblies 204. The initial slots 223 may include a depth extending radially inward from the outer surface 225 for receiving retractable portions of the handle assemblies 204. The initial slots 223 may include a length extending axially parallel along the outer surface 225. The initial slots 223 may include a width extending along a portion of the circumference of the outer surface 225.

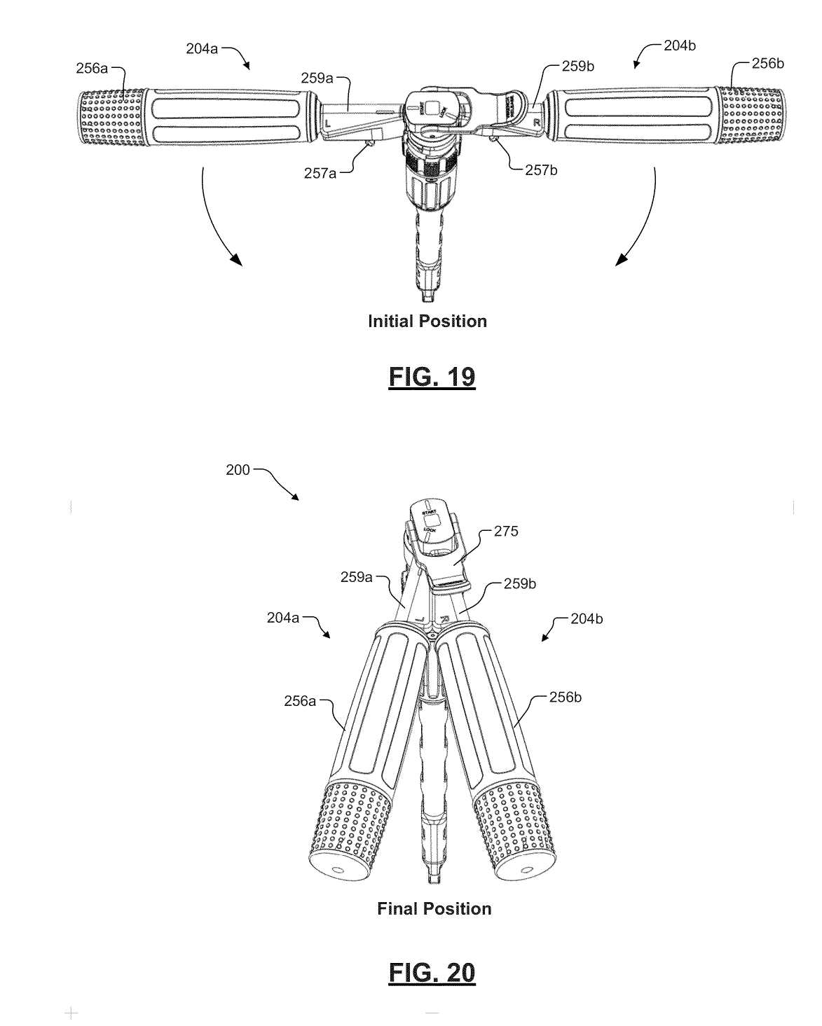

For example, first initial slot 223a may extend from the proximal surface 221 distally a first length L1 along the outer surface 225 of the receiving block 215. First initial slot 223a may terminate at the first ledge 227a which extends circumferentially around at least a portion of the outer surface 225 of the receiving block 215. The first handle assembly 204a includes features that engage the first initial slot 223a. The first handle assembly 204a may engage the first initial slot 223a when the instrument 200 is in a non-deployed configuration or an initial configuration prior to deploying the blades 304 and 306 of the instrument as illustrated in FIG. 19. The initial slots 223 limit rotation of the first handle assembly 204a in a first direction, such as a clockwise direction as described below.

The receiving block 215 further includes first and second deployed slots 229a and 229b (collectively deployed slots 229) that engage features of at least one of the handle assemblies 204. The deployed slots 229 may include depth, width, and length similar to the initial slots 223. For example, first deployed slot 229a may extend from the proximal surface 221 distally the first length L1 along the outer surface 225 of the receiving block 215. First deployed slot 229a may terminate at the first ledge 227. The first handle assembly 204a includes features that engage the first deployed slot 229a. The first handle assembly 204a may engage the first deployed slots 229a when the instrument 200 is in a deployed configuration or a final configuration after deploying the blades 304 and 306 of the implant 300 as illustrated in FIG. 20. The deployed slots 229 limit rotation of the first handle assembly 204a in a second direction, such as a counter-clockwise direction as described below.

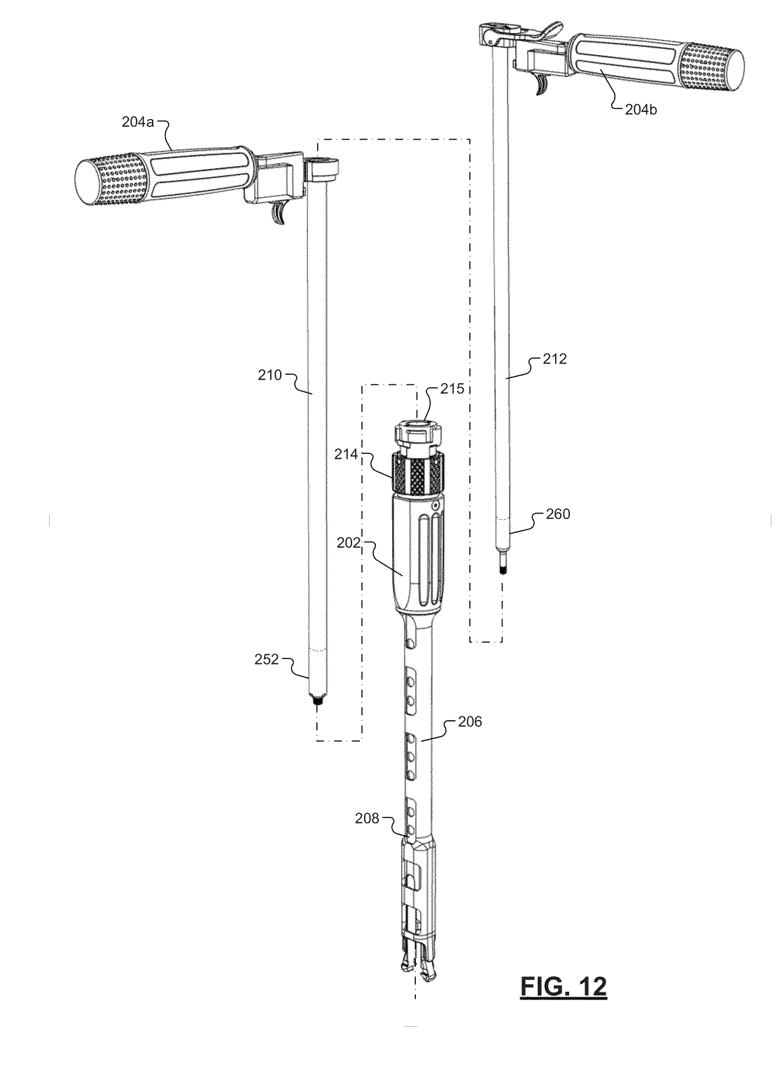

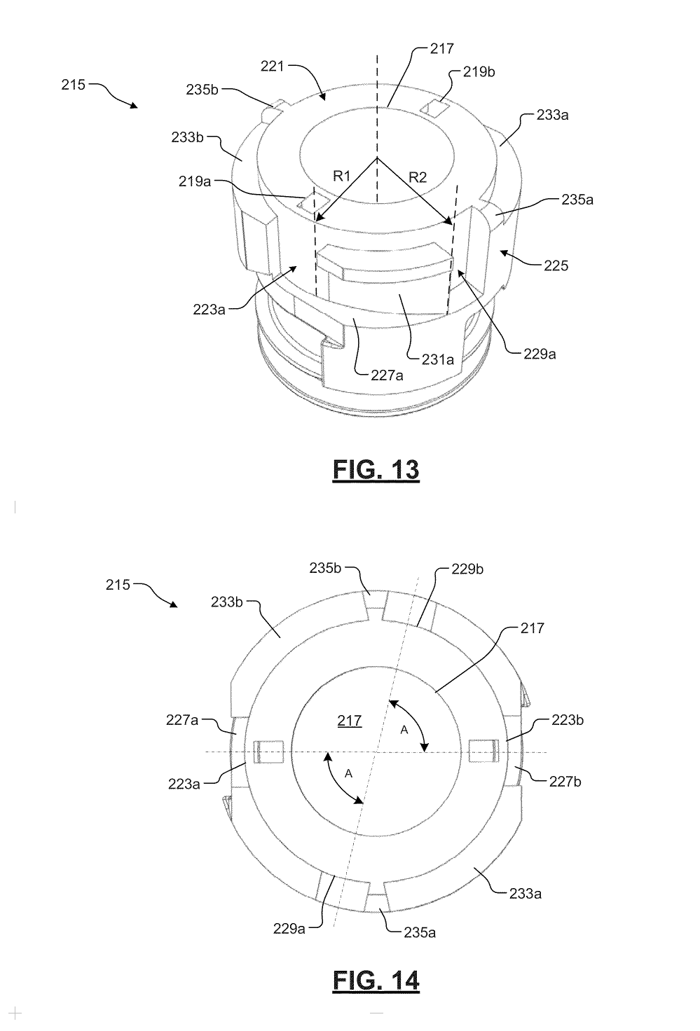

The slots 223 and 229 may be disposed along the circumference of the receiving block 215 at various angles to control an amount of deployment of at least one of the blades 304 and 306 of the implant 300. The initial slots 223, the first ledge 227, and the deployed slots 229 may engage portions of the first handle assembly 204a to limit rotation of the handle assembly 204a. For example, initial slot 223a may be separated from a corresponding deployed slot 229a by an angle A correlated to the amount of rotation required to deploy a first blade 304. The outer surface 225 may further include radially ramped portions 231 extending from each initial slot 223 to each corresponding deployed slot 229. The radially ramped portions 231 include an increasing radius. For example, ramped portion 231a may include a first radius R1 of outer surface 225 corresponding to a depth of the initial slot 223a and increase to a second radius R2 just before the deployed slot 229a as shown in FIGS. 13 and 14. The ramped portions 231 provide increasing resistance on a feature of the first handle assembly 204a as it rotates from the initial slot 223 to the deployed slot 229. The increasing resistance may provide tactile feedback to the user. The increasing radius may cause a feature of the first handle assembly 204a to snap into the deployed slot 229a providing audible feedback.

The receiving block 215 includes additional features that may engage with the second handle assembly 204b. The receiving block 215 may include a second ledge 233 and first and second stops 235a and 235b (collectively stops 235). For example, the second ledge 233 may be proximally disposed from the first ledge 226 a distance L2 and engage with features of the second handle assembly 204b to limit distal travel of the inner shaft 206. The stops 235 may be disposed along the second ledge 233 and limit rotation of the second handle assembly 204b. The second handle assembly 204b includes features that engage the second stop 235b. The second handle assembly 204b may engage the second stop 235b when the instrument 200 is in a deployed configuration or a final configuration after deploying the blades 304 and 306 of the implant 300 as illustrated in FIG. 20. The stops 235 limit rotation of the second handle assembly 204b in a first direction, such as a clockwise direction as described below.

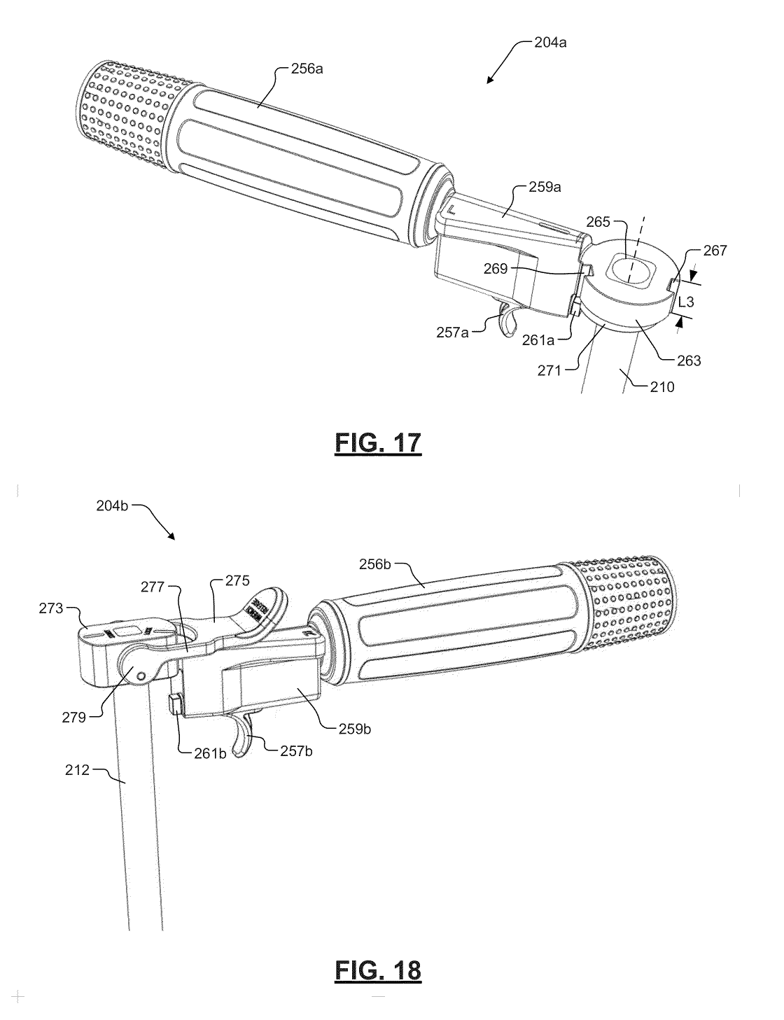

Referring now to FIGS. 17 and 18, the first and second handle assemblies 204a and 204b include similar features such as first and second handles 256a/b (collectively handles 256), triggers 257a/b (collectively triggers 257), and housings 259a/b (collectively housings 259) that engage various features of the handle assemblies 204 and the receiving block 215. The handles 256 may include a gripping surface such as a silicone wrap or other textured surface to enhance the user's grip on the instrument 200. The housings 259 may house the triggers 257, each of which may include first and second projections 261a/b (collectively projections 261) extending outside the housings 259. The triggers 257 may be biased by one or more bias mechanisms such as a spring to extend the projections 261 away from proximal ends of the housings 259.

In FIG. 17, the first handle assembly 204a includes first trigger 257a with first projection 261a extending away from first housing 259a for engaging initial slots 223, first ledge 227, ramped portions 231, and deployed slots 229. The first housing 259a may be rigidly coupled with a receiving end 263 of the outer shaft 210 and extend radially way from the outer shaft 210. The receiving end 263 includes an aperture 265 through which the inner shaft 210 may be inserted. The receiving end 263 may include a circular outer profile with one or more notches and pathways about the perimeter for receiving features of the second handle assembly 204b.

For example, a first notch 267 may be disposed at a point along the circumference of the receiving end 263 that is diametrically opposite of point of attachment of the first housing 259a. When the first handle assembly 204a is inserted into the receiving block 215 and aligned with the first marking 219a, the first notch 267 may be aligned with the second marking 219b. The first notch 267 may extend from a proximal side of the receiving end 263 to a distal side that engages the proximal surface of the receiving block 215. For example, the first notch 265 may extend a length L3. A second notch 269 may be disposed at a point along the circumference of the receiving end 263 that is proximate to the point of attachment of the first housing 259a. For example, the second notch 269 may be disposed at some angle relative to the first housing 259a and/or the first notch 267. The first and second notches 267 and 269 may also extend the length L3. A radially ramped portion 271 may extend between the first notch 267 and the second notch 269 similar to the increasing radius of radially ramped portion 231 of the receiving block 215 to provide tactile and audible feedback similar to the slots 223 and 229 above.

In FIG. 18, the second handle assembly 204b includes second trigger 257b with second projection 261b extending away from the second housing 259b for engaging the first notch 267, second notch 269, and radially ramped portion 271. The second housing 259b may be coupled with a top portion 273 of the inner shaft 212. A release mechanism 275 may be pivotally coupled to the top portion 273. The release mechanism 275 may include a lever arm 277 with a cam portion 279 that engages the receiving end 263 of the outer shaft 210. As the lever arm 277 rotates, the cam portion 279 forces the top portion 273 of the inner shaft 212 away from the receiving end 263 of the outer shaft 210 to separate the surfaces therebetween.

FIGS. 19 and 20 illustrate actuation of the instrument 200 for deploying features of the implant 300. Initially, the first handle assembly 204a and second handle assembly 204b may be diametrically opposed 180 degrees to form a generally T-shaped configuration in an initial position. The first projection 261a of the first handle assembly 204a may be aligned with the first initial slot 223a. The second projection 261b of the second handle assembly 204b may be aligned with the first notch 267 of the receiving end 263 of the outer shaft 210. The user may apply torque to the handles 256a and 256b causing the inner and outer shafts 210 and 212 to counter-rotate about their common axis. As the handle assemblies 204 rotate towards one another, the first projection 261a may engage along the ramped portion 231 of the receiving block 215 and the second projection 261b may engage along the ramped portion 271 of the receiving end 263 of the outer shaft 210.

When the handle assemblies 204 are fully rotated, the instrument may form a more generally V-shaped appearance viewed from the proximal end in a final position. Upon full deployment of the features of the implant 300, the first projection 261a may engage the first deployed slot 229a. The second projection 261b may engage the stop 235 of the receiving block as well as the second notch 269. The projections 261 may snap into place due to force applied by a bias member. Thus, the instrument 200 may provide tactile and audible feedback when full deployment of the features of the implant are complete. Upon deployment, the instrument 200 may be removed by actuating the release mechanism 275 to pry apart the second handle assembly 204b from the first handle assembly 204a and thus, to separate the inner shaft 212 from the implant 300. The outer shaft 210 may subsequently be removed from the implant 300.

Although the instrument 200 is described with reference to the sleeve 206, clamping member 208, and alignment block 215, the features of the implant 300 may be deployed using only the handle assemblies 204 and their respective shafts 210 and 212. For example, the implant 300 may be inserted into the disc space between adjacent vertebrae using any of a variety of instruments. Once within the disc space, the additional diameter of the sleeve 206 may create difficulty in maneuvering the instrument 200 within the patient, especially in lower regions of the spine near the pelvic bone. The shafts 210 and 212 may be inserted directly into the implant 300 to deploy the blades 304 and 306. Surfaces of the handles 204a and 204b may prevent some over deployment. However, ideally, the full system including the alignment block 215 would be used to prevent uniform deployment of the blades 304 and 306.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

The invention claimed is:

1. A surgical instrument for inserting a spinal implant comprising:
an inserter portion including at a proximal end a receiving block, at a distal end a sleeve, and a channel extending therethrough;
a clamping member including at a proximal end movably coupled with the receiving block, a middle portion that slides within the channel, a distal end that couples the spinal implant, and a first axial bore extending therethrough;
a first actuator including a proximal end with a first projection for engaging the receiving block, a first shaft extending through the first axial bore, and a distal end with a first engagement feature for engaging a first deployment feature of the implant; and
a second actuator including a proximal lend with a second projection for engaging the receiving block, a second shaft extending through a second axial bore of the first shaft, and a distal end with a second engagement feature for engaging a second deployment feature of the implant.

2. The surgical instrument of claim 1, wherein the receiving block includes features for limiting rotational movement of at least one of the first actuator and the second actuator.

3. The surgical instrument of claim 1, wherein the receiving block includes an initial slot on the circumferential surface at a first radius from a longitudinal axis of the instrument.

4. The surgical instrument of claim 3, wherein the initial slot transitions to a ramped portion of increasing radius.

5. The instrument of claim 4, wherein the ramped portion terminates at a second radius that is great than the first radius.

6. The instrument of claim 5, wherein the ramped portion transitions to a deployed slot at a radius less than the second radius.

7. The instrument of claim 6, wherein the first projection is configured to engage at least one of the initial slot, the ramped portion, and the deployed slot during actuation of the first deployment feature of the implant.

8. The instrument of claim 1, wherein the proximal end of the first actuator includes a receiving end with features for limiting rotational movement of at least one of the first actuator and the second actuator.

9. The instrument of claim 8, wherein the receiving end includes a first notch on the circumferential surface at a first angle relative to a first handle assembly.

10. The instrument of claim 9, wherein the receiving end includes a second notch disposed at a second angle relative to the first handle assembly.

11. The instrument of claim 10, wherein the receiving end includes a ramped portion between the first notch and the second notch.

12. The instrument of claim 1, further comprising a release mechanism that forces the first actuator and the second actuator apart.

13. The instrument of claim 12, wherein the release mechanism includes a lever arm and a cam portion coupled with the second actuator such that rotation of the lever arm applies force through the cam portion on the first actuator.

14. The surgical instrument of claim 1, wherein the coupling member includes a pair of arms at the distal end for clamping the spinal implant.

15. The surgical instrument of claim 1, wherein the first actuator rotates in a first direction to deploy the first deployment feature and the second actuator rotates in a second direction to deploy the second deployment feature.

16. The surgical instrument of claim 1, wherein the distal end of the first shaft and the distal end of the second shaft includes at least one of a splined projection and a hexagonal projection.

17. A system for a spinal procedure comprising:
an implant including a first deployment feature and a second deployment feature configured to engage with vertebrae; and
an instrument including
an inserter portion including at a proximal end a receiving block, at a distal end a sleeve, and a channel extending therethrough;
a coupling member including at a proximal end movably coupled with the receiving block, a middle portion that slides within the channel, a distal end that couples the spinal implant, and a first axial bore extending therethrough;
a first actuator including a proximal end with a first projection for engaging the receiving block, a first shaft extending through the first axial bore, and a distal end with a first engagement feature for engaging a first deployment feature of the implant; and
a second actuator including a proximal lend with a second projection for engaging the receiving block, a second shaft extending through a second axial bore of the first shaft, and a distal end with a second engagement feature for engaging a second deployment feature of the implant.

18. The system of claim 17, wherein the receiving block includes features for limiting rotational movement of at least one of the first actuator and the second actuator to limit actuation of at least one of the first deployment feature and the second deployment feature.

19. The system of claim 17, wherein the proximal end of the first actuator includes a receiving end with features for limiting rotational movement of at least one of the first actuator and the second actuator.

20. The system of claim 17, further comprising a release mechanism that forces the first actuator and the second actuator apart.
